(19) United States
(12) Patent Application Publication
(10) Pub. No.: US 2024/0366644 A1
(43) Pub. Date: Nov. 7, 2024

(54) ANTI-INFLAMMATORY APPLICATION OF GINSENOSIDE RD

(71) Applicant: Li FU, Liaoning (CN)

(72) Inventors: Li FU, Liaoning (CN); Dayun SUI, Liaoning (CN); Huali XU, Liaoning (CN); Mingming LU, Liaoning (CN); Xiaofeng YU, Liaoning (CN); Shuo WANG, Liaoning (CN); Wenwen FU, Liaoning (CN); Xue FENG, Liaoning (CN); Yuangeng LI, Liaoning (CN); Baohua SUN, Liaoning (CN); Wenfei FU, Liaoning (CN); Rongxin LIN, Liaoning (CN); Yang LIU, Liaoning (CN); Xin GE, Liaoning (CN)

(21) Appl. No.: 18/768,402

(22) Filed: Jul. 10, 2024

Related U.S. Application Data
(63) Continuation-in-part of application No. PCT/CN2022/143825, filed on Dec. 30, 2022.

(30) Foreign Application Priority Data
Jan. 11, 2022 (CN) 202210027815.3

(51) Int. Cl. A61K 31/704; A61K 9/00; A61K 9/06; A61K 9/20; A61K 9/48; A61K 31/375; A61K 36/539; A61K 36/64; A61K 36/752; A61K 36/8905; A61K 36/8965; A61K 36/8966; A61P 11/00; A61P 11/06; A61P 29/00

(52) U.S. Cl. CPC A61K 31/704; A61K 9/0053; A61K 9/06; A61K 9/2009; A61K 9/2013; A61K 9/2059; A61K 9/4866; A61K 31/375; A61K 36/539; A61K 36/64; A61K 36/752; A61K 36/8905; A61K 36/8965; A61K 36/8966; A61P 11/00; A61P 11/06; A61P 29/00

(57) ABSTRACT

The present invention discloses a novel application of ginsenoside Rd in preparation of a drug for relieving or/and treating inflammations and health food/medicinal and edible food/functional food/dietary supplement. Tests prove that ginsenoside Rd has a remarkable curative effect on inflammations, takes effect quickly, has a better effect than Dexamethasone, is suitable for both acute inflammations such as influenza virus pneumonia, novel coronavirus pneumonia and acute lung injury inflammations, and chronic inflammations such as chronic obstructive pneumonia and chronic asthmatic airway inflammations, and is regarded as a new drug source for treating acute and chronic inflammations and complications thereof.

### CROSS-REFERENCE TO RELATED APPLICATIONS

This application is a continuation-in-part application of PCT/CN2022/143825 having the international filing date of Dec. 30, 2022 and claiming the benefit of priority to Chinese patent application CN 202210027815.3 with the filing date of Jan. 11, 2022, the entire disclosure of both applications is incorporated by reference herein.

### TECHNICAL FIELD

The present invention relates to the technical field of medicine, relates to an anti-inflammatory drug or health food/medicinal and edible food/functional food/dietary supplement, and more particularly, relates to an application of ginsenoside Rd in preparation of an anti-inflammatory drug or health food/medicinal and edible food/functional food/dietary supplement.

### BACKGROUND

Inflammation is a protective response of the body against external infections or noxious stimulation, and is also a common pathological process of many different types of diseases. Long-term persistent and excessive inflammatory response or inflammatory storm will cause serious damages to the body itself, and even organ failure, so drug intervention is required to block the continuation and development of inflammations. At present, there are two main types of anti-inflammatory drugs commonly used in clinical practice: non-steroidal anti-inflammatory drugs and steroidal anti-inflammatory drugs (glucocorticoids). These two types of anti-inflammatory drugs have good clinical anti-inflammatory effects, but will produce a series of adverse reactions and tolerances after long-term massive use, e.g., the former causes gastric mucosal injuries, liver and kidney damages and cardiac toxic and side effects, and the latter causes water and sodium retention. In order to solve the tolerances and adverse reactions of drugs, the search for anti-inflammatory drugs with novel structures and unique mechanisms has always been a hot topic in the field of research and development of novel anti-inflammatory drugs. Ginsenoside Rd belongs to a diol type of ginsenoside, which is mainly found in Radix Ginseng, Radix Notoginseng, and Radix Panacis Quinquefolii. It has a wide range of biological activities, obvious pharmacological effects on heart and cerebral vessels, nervous systems, immune systems, etc., as well as the effects better than other monomeric saponins in analgesia and neuroprotection, but its anti-inflammatory and protective effects and mechanisms against acute inflammation (ALI) have not been reported in literatures.

### SUMMARY

A primary objective of the present invention is to provide anti-inflammatory properties and efficacy of ginsenoside Rd in view of the technical problems existing in the above-mentioned existing anti-inflammatory drugs or health food/medicinal and edible food/functional food/dietary supplement, and to provide new medicinal use of the ginsenoside Rd, that is, a novel application of the ginsenoside Rd in an anti-inflammatory drug or health food/medicinal and edible food/functional food/dietary supplement.

To fulfill said objective, in one aspect, the present invention provides an application of ginsenoside Rd in preparation of an anti-inflammatory drug or health food/medicinal and edible food/functional food/dietary supplement.

In the process of screening natural active ingredients with anti-inflammatory effects, the inventors have found that ginsenoside Rd in the chemical composition of Radix Ginseng has a significant anti-inflammatory effect.

The drug is used for the treatment of one or more of acute inflammations and chronic inflammations.

The drug or health food/medicinal and edible food/functional food/dietary supplement is composed of ginsenoside Rd and a pharmaceutically acceptable carrier.

The content of the ginsenoside Rd is 1% to 99.9%; preferably, 30 to 99.9%; further preferably, 60 to 99.9%; further preferably, 80 to 99.9%; and preferably, 98 to 99.9%. In particular, a ratio of a weight of the ginsenoside Rd to a total weight of the preparation is (0.01-100): 100, preferably (0.1-100): 100, and further preferably (10-100): 100.

In particular, the pharmaceutically acceptable carrier is often recognized by health professionals for this objective and as an inactive ingredient in pharmaceuticals. The compilation related to pharmaceutically acceptable carriers can be found in handbooks such as “Handbook of Pharmaceutical Excipients” (2nd Edition, edited by A. Wade and P. J. Weller; published by American Pharmaceutical Association, Washington and The Pharmaceutical Press, London, 1994).

In particular, the carrier includes an excipient, such as starch or water; a lubricant, such as magnesium stearate; a disintegrant, such as microcrystalline cellulose; filler, such as lactose; a binder, such as pregelatinized starch or dextrin; a sweetener; an antioxidant; an ointment base; a transdermal promoter; a preservative; a flavoring agent; spices, etc.; and the drug is an oral preparation, a topical preparation or an injection; the oral preparation is tablets, capsules, granules, pills, a solution, a suspension, an emulsion, an oral liquid, drops, or a syrup; the topical preparation is emplastrum, an ointment or a suppository; and the injection is an injection solution, a powder injection or a lyophilized powder injection.

The health food/medicine and edible food/functional food/dietary supplement is an oral preparation; and the oral preparation is tablets, a powder, granules, medicinal tea, pills, a paste, hard capsules, soft capsules, an oral liquid, medicated wine, a syrup, or drops. In another aspect, the present invention provides an anti-inflammatory drug or health food/medicinal and edible food/functional food/dietary supplement, wherein the drug or health food/medicinal and edible food/functional food/dietary supplement contains ginsenoside Rd.

The content of the ginsenoside Rd is 1% to 99.9%; preferably, 30 to 99.9%; further preferably, 60 to 99.9%; further preferably, 80 to 99.9%; and preferably, 98 to 99.9%. In particular, a ratio of a weight of the ginsenoside Rd to a total weight of the preparation is (0.01-100): 100, preferably (0.1-100): 100, and further preferably (10-100): 100. In particular, the drug or health food/medicinal and edible food/functional food/dietary supplement further includes one or more of a Radix Scrophulariae extract, a Rehmannia glutinosa Libosch extract, a Radix Ophiopogonis extract, a Radix Scutellariae extract, a Radix Ranunculi Ternati extract, a Fritillaria extract, a Menthae Haplocalycis Herba extract, a Semen Sesami Nigrum extract, a Zingiber officinale Roscoe extract, a grape seed extract, a pomegranate seed extract, a Rhizoma Cyperi extract, a Pericarpium Citri Reticulatae Viride extract, plant essential oil, arbutin, vitamin C and derivatives thereof, or vitamin E and derivatives thereof.

The drug may be prepared into various dosage forms by methods well-known in the art, such as tablets, capsules, pills, powder, granules, a syrup, emplastrum, an ointment, or a suppository.

Compared with the prior art, the present invention has the following obvious advantages.
1. The present invention has discovered a new medicinal value for the known compound ginsenoside Rd, which is used for an anti-inflammatory purpose, and can be prepared into an anti-inflammatory drug or health food/medicinal and edible food/functional food/dietary supplement, thus opening up a new field for the application of Radix Ginseng medicinal materials.
2. A series of experimental studies of the present invention prove that the ginsenoside Rd has a significant anti-inflammatory effect.
3. The ginsenoside Rd of the present invention has a strong anti-inflammatory pharmacological effect, takes effect quickly, has small toxic and side effects and good safety, can be taken for a long time, and has a good medicinal prospect.
4. The product of the present invention is abundant and cheap in raw materials, safe for clinical use, and simple in preparation process, can be prepared into various dosage forms, and is small in dosage, convenient to use, and thus easy to promote.
5. According to the present invention, the single-component ginsenoside Rd (an anti-inflammatory active ingredient) can be used to prepare an anti-inflammatory drug, or a composite drug for the treatment of inflammations can also be prepared by a combined formulation of the ginsenoside Rd and other active ingredients (e.g., a Radix Notoginseng, a Radix et Rhizoma Tripterygii extract, a Radix Bupleuri extract, a powered Radix Stephaniae Tetrandrae extract, a Radix Scutellariae extract, a Radix Sophorae Flavescentis extract, a Melilotus suaveolens Ledeb extract, a grape seed extract, a pomegranate seed extract, vitamin C and its derivatives, or vitamin E and its derivatives).

### DETAILED DESCRIPTION

The beneficial effects of the formula of the present invention are further described below by specific embodiments, and these examples include pharmacodynamic tests for capsules, tablets and soft capsules of ginsenoside Rd of the present invention. These examples are only exemplary and do not constitute any limitation on the scope of the present invention. Those skilled in the art should understand that the details and forms of the technical solutions of the present invention may be modified or replaced without deviating from the formula idea and the use scope of the present invention, but these modifications and substitutions fall within the protection scope of the present invention.

### Example 1 Ginsenoside Rd Tablets

Ginsenoside Rd tablets were prepared in the following proportions:

| | |
|---|---|
| Ginsenoside Rd (98% in content) | 500 g |
| Starch | 1000 g |
| Talcum powder | 1% (15 g) |
| Magnesium stearate | 1% (15 g) |

Ginsenoside Rd and starch were mixed well and prepared into granules, and the granules were added with talcum powder and magnesium stearate, mixed well and pressed into 10,000 tablets.

### Example 2 Ginsenoside Rd Capsules

1. Raw Materials were Prepared According to the Following Proportions:

| | |
|---|---|
| Ginsenoside Rd (98% in purity) | 500 g |
| Starch | 1000 g |

Ginsenoside Rd and starch were mixed well and packed into 10,000 capsules.

### Example 3 Ginsenoside Rd Granules

1. Raw Materials were Prepared According to the Following Proportions:

| | |
|---|---|
| Ginsenoside Rd (63% in purity) | 10 g |
| Starch | 1000 g |

Ginsenoside Rd and starch were mixed well and prepared into granules, which were then loaded into 10,000 bags.

### Example 4 Ginsenoside Rd Tablets

1. Raw Materials were Prepared According to the Following Proportions:

| | | |
|---|---|---|
| Ginsenoside Rd (63% in purity) | 30 | g |
| Rehmannia glutinosa Libosch extract | 20 | g |
| Radix Ophiopogonis extract | 40 | g |
| Starch | 1000 | g |

| | |
|---|---|
| Talcum powder | 1% (14.5 g) |
| Magnesium stearate | 1% (14.5 g) |

Ginsenoside Rd, Rehmannia glutinosa Libosch extract, Radix Ophiopogonis extract and starch were mixed well and prepared into granules, and the granules were added with talcum powder and magnesium stearate, mixed well and pressed into 10,000 tablets.

### Example 5 Ginsenoside Rd Capsules

1. Raw Materials were Prepared According to the Following Proportions:

| | | |
|---|---|---|
| Ginsenoside Rd (63% in purity) | 30 | g |
| Fritillaria extract | 30 | g |
| Vitamin C | 600 | g |
| Starch | 1000 | g |

Ginsenoside Rd, Fritillaria extract, Vitamin C and starch were mixed well and packed into 10,000 capsules.

### Example 6 Ginsenoside Rd Granules

| | |
|---|---|
| Ginsenoside Rd (63% in purity) | 30 g |
| Radix Scutellariae extract | 30 g |
| Vitamin C | 600 g |
| Powdered sucrose | 5000 g |

Ginsenoside Rd, Radix Scutellariae extract, Vitamin C and powdered sucrose were mixed well and prepared into granules, which were then loaded into 10,000 bags.

### Example 7 Ginsenoside Rd Oral Liquid

| | |
|---|---|
| Ginsenoside Rd (63% in content) | 4% |
| Rhizoma Cyperi extract | 3% |
| Pericarpium Citri Reticulatae | 3% |
| Viride extract | |
| Glucose syrup | 5% |

Deionized water was finally added to the whole formula to 100%. Ginsenoside Rd, Rhizoma Cyperi extract and Pericarpium Citri Reticulatae Viride extract were dissolved with ethanol, and added with glucose syrup to 100% to obtain the finished product.

### Example 8 Ginsenoside Rd Solid Drink

| | |
|---|---|
| Ginsenoside Rd (50% in purity) | 30 g |
| Radix Scutellariae extract | 30 g |
| Vitamin C | 600 g |
| Powdered sucrose | 5000 g |

Ginsenoside Rd, Radix Scutellariae extract, Vitamin C and powdered sucrose were mixed well and prepared into granules, which were then loaded into 10,000 bags.

### Example 9 Ginsenoside Rd Tableted Candies

| | |
|---|---|
| Ginsenoside Rd (10% in purity) | 600 g |
| Xylitol | 350 g |
| Citric acid | 10 g |
| Aspartame | 15 g |
| Magnesium stearate | 10 g |
| Menthae Haplocalycis | 15 g |
| Herba essence | |

Ginsenoside Rd, xylitol, citric acid, aspartame and Menthae Haplocalycis Herba essence were mixed well and prepared into granules, and the granules were added with magnesium stearate, mixed well and pressed into 1000 tablets.

### Example 10 Ginsenoside Rd Ointment

| | |
|---|---|
| Ginsenoside Rd | 100 g |
| (63% in content) | |
| Aqueous phase | 250 g |
| (methylparaben, water) | |
| Oil phase | 1000 g |
| (octadecanol, vaseline) | |

The aqueous phase was added to the oil phase, stirred, and added with ginsenoside Rd to obtain the finished product.

### Experimental Example 1 Effect of Ginsenoside Rd on Xylene-Induced Swelling of Mouse Ears

### 1.1 Experimental Animals and Pharmaceuticals

### 1.1.1 Experimental Animals:

Male and female Kunming mice (SPF grade), weighing 18-22 g, were purchased from Liaoning Changsheng Biotechnology Co., Ltd., with quality certificate number: SCXK-(Liaoning) 2020-0001.

### 1.1.2 Reagents and Tested Drugs:

Dexamethasone, purchased from Shanghai Chemical Reagent Company;
Indomethacin capsules, produced by CSPC Pharmaceutical Group Hebei Yongfeng Pharmaceutical Co., Ltd.; and
ginsenoside Rd (content ≥95%), produced by Dalian Fusheng Natural Medicine Development Co., Ltd., detected by HPLC with Ginsenoside Rd provided by China Institute for Food and Drug Control as a reference substance, and having a content of 96.2%.

### 1.2 Experimental Method

Twenty KM mice, weighing 18-22 g, were randomly grouped, with ten mice in each group. The groups include a model group, a positive control group (Indomethacin, 10 mg/kg), a ginsenoside Rd low-dose group (5 mg/kg), and a ginsenoside Rd high-dose group (10 mg/kg). After 30 min of gavage administration of mice (the model group was administrated with the equal volume of 0.5% sodium carboxymethylcellulose by gavage), 0.03 ml of xylene was evenly applied to both sides of the right ear of each mouse in each group to cause inflammations on both sides, and the left ear of the mouse was not applied as a control. After 4 h, the mouse was killed by dislocation of cervical vertebra, the two ears of the mouse were cut off along the auricle, round ear slices of the same size (a diameter of 6 mm) were taken at the same position of the left and right ears and weighed respectively, a mass difference of the two ear slices was used as an ear swelling degree, and a swelling inhibition rate was calculated.

$$Ear swelling rate ( \% ) = [ right ear weight ( swollen ear ) - left ear weight ( control ear ) ] / left ear weight ( control ear ) \times 100 \% .$$

$$Swelling inhibition rate ( \% ) = [ ( ear swelling rate in the model group - ear swelling rate in the test group ) / ear swelling rate in the model group ] \times 100 \% .$$

### 1.3 Results and Conclusions

TABLE 1-1
The results of ear mass difference, ear swelling rate and
ear swelling inhibition rate of mice in various groups

| | | Ear mass | Swelling | Inhi- |
|---|---|---|---|---|
| | | difference | degree | bition |
| Group | Dose | (mg) | (mg) (%) | rate (%) |
| Model group | — | 16.37 ± 1.17 | 10.85 ± 1.73 | — |
| Indomethacin | 10 mg/kg | 10.87 ± 0.53 | 6.48 ± 1.89 | 40.28 |
| group | | | | |
| Ginsenoside Rd | 5 mg/kg | 11.84 ± 1.02 | 7.56 ± 2.05 | 30.32 |
| low-dose | | | | |
| group | | | | |
| Ginsenoside Rd | 10 mg/kg | 7.68 ± 0.39** | 6.59 ± 1.96 | 39.26 |
| high-dose | | | | |
| group | | | | |

Compared with a positive drug group,
**P < 0.01

Compared with the model group, the ginsenoside Rd drug group could significantly inhibit ear swelling of mice caused by xylene, and had a significant anti-inflammatory effect. The ear swelling inhibition rate at a dose of 10 mg/kg was close to that of Indomethacin, achieving a more obvious anti-inflammatory effect.

### Experimental Example 2 Effect of Ginsenoside Rd on Pneumonia Caused by Mouse Influenza Virus

### 1.1 Experimental Animals and Pharmaceuticals

### 1.1.1 Experimental Animals:

ICR mice, weighing 20-30 g, laboratory animal supplier: Hunan SJA Laboratory Animal Co., Ltd., laboratory animal production license: SCXK (Xiang) 2019-0004, and feed supplier: Jiangsu Medicience Biopharmaceutical Co., Ltd.

### 1.1.2 Virus and Drug:

Influenza virus (BSL-3 Laboratory, Guangzhou Customs Technology Center (Laboratory of Highly Pathogenic Microorganisms, State Key Laboratory of Respiratory Diseases). Ginsenoside Rd (content ≥95%), produced by Dalian Fusheng Natural Medicine Development Co., Ltd., detected by HPLC with Ginsenoside Rd provided by China Institute for Food and Drug Control as a reference substance, and having a content of 96.2%.

### 1.2 Experimental Method

### (1) Modeling

A lump of absorbent cotton was put into a beaker of 200-300 mL, and then poured with an appropriate amount of ether (till the absorbent cotton was wetted). The beaker filled with the absorbent cotton was inverted, and added with the mice for anesthesia. When the mice were extremely excited and then obviously weak, that is, the anesthesia depth was moderate, the mice were taken out. The ether-anesthetized mice were placed on their backs, with the mouse head facing upward, and then were subjected to intranasal infection. A diluted influenza virus was added to nostrils at 0.03 mL/nostril. A blank group was administrated with normal saline, instead of a virus suspension.

### (2) Grouping and Administration

The mice were randomly divided into four groups, that is, drug groups (10 mg/kg, 5 mg/kg), a model group, and a blank group were administrated by routine gavage on the day before infection, once a day; and the blank group and the model group were perfused with the equal volume of normal saline.

### (3) TNF-α and IL-6 Detection in Serum

On the Day 1, Day 3, Day 5 and Day 7 after influenza virus infection, venous blood was collected from the plexus ophthalmicus, and blood from mice in each group was preserved, subjected to serum separation, and stored in a refrigerator for later use.

According to the instructions of a test kit, various detection and analysis were carried out, and TNF-α and IL-6 concentrations were calculated based on the standard detection and standard curve plotting.

### (4) TNF-α and IL-6 Detection in Lung Tissues

On the Day 1, Day 3, Day 5 and Day 7 after influenza virus infection, after the mice were killed by bleeding from the eyeballs, the whole lungs were picked, homogenized in a homogenizer according to a ratio of lung weight (g):normal saline (mL)=1:9, and centrifuged; and a supernatant was stored in the refrigerator for later use. According to the instructions of a test kit, various detection and analysis were carried out, and TNF-α and IL-6 concentrations were calculated based on the standard detection and standard curve plotting.

### 1.3. Test Results

### (1) Effect of Ginsenoside Rd on TNF-α Content in Serum of Influenza Virus-Infected Mice

TABLE 2-1
Effect of ginsenoside Rd on TNF-α content in serum of influenza
virus-infected mice (x̅ ± s, n = 10)

| | Serum TNF-α (pg/mL) | | | |
|---|---|---|---|---|
| Group | 1 d | 3 d | 5 d | 7 d |
| Blank group | 25.47 ± 1.65 | 26.05 ± 1.37 | 25.01 ± 1.08 | 24.97 ± 1.26 |
| Model group | 29.61 ± 1.07 | 29.42 ± 1.25 | 28.74 ± 1.35 | 28.21 ± 1.27 |
| Ginsenoside Rd | 28.32 ± 1.21 | 28.19 ± 1.27 | 28.07 ± 1.43 | 27.94 ± 1.16 |
| low-dose | | | | |
| group | | | | |
| Ginsenoside Rd | 26.53 ± 1.02 | 26.83 ± 2.04 | 26.99 ± 1.55 | 25.88 ± 1.66 |
| high-dose | | | | |
| group | | | | |

### (2) Effect of Ginsenoside Rd on TNF-α Content in Lung Tissues of Influenza Virus-Infected Mice

TABLE 2-2
Effect of ginsenoside Rd on TNF-α content in lung tissues of influenza
virus-infected mice (x̅ ± s, n = 10)

| | Lung tissue TNF-α (pg/mL) | | | |
|---|---|---|---|---|
| Group | 1 d | 3 d | 5 d | 7 d |
| Blank group | 29.68 ± 1.85 | 29.28 ± 2.28 | 29.53 ± 2.80 | 29.26 ± 2.16 |
| Model group | 33.76 ± 2.39 | 35.43 ± 3.48 | 34.07 ± 2.53 | 32.84 ± 2.46 |
| Ginsenoside Rd | 32.98 ± 2.27 | 34.72 ± 2.44 | 32.14 ± 2.16 | 30.86 ± 3.05 |
| low-dose | | | | |
| group | | | | |
| Ginsenoside Rd | 31.34 ± 3.25 | 32.09 ± 2.51 | 31.97 ± 2.94 | 30.08 ± 3.28 |
| high-dose | | | | |
| group | | | | |

### (3) Effect of Ginsenoside Rd on IL-6 Content in Serum of Influenza Virus-Infected Mice

TABLE 2-3
Effect of ginsenoside Rd on IL-6 content in serum of influenza
virus-infected mice (x̅ ± s, n=10)

| | Serum IL-6 (pg/mL) | | | |
|---|---|---|---|---|
| Group | 1 d | 3 d | 5 d | 7 d |
| Blank group | 45.57 ± 1.96 | 44.47 ± 2.93 | 45.83 ± 2.52 | 45.15 ± 2.37 |
| Model group | 48.39 ± 2.63 | 48.34 ± 3.01 | 47.35 ± 2.49 | 47.48 ± 3.08 |
| Ginsenoside Rd | 47.15 ± 2.31 | 47.22 ± 2.57 | 46.62 ± 2.01 | 46.59 ± 3.46 |
| low-dose | | | | |
| group | | | | |
| Ginsenoside Rd | 46.21 ± 3.03 | 46.52 ± 2.23 | 45.79 ± 3.19 | 45.23 ± 2.95 |
| high-dose | | | | |
| group | | | | |

### (4) Effect of Ginsenoside Rd on IL-6 Content in Lung Tissues of Influenza Virus-Infected Mice

TABLE 2-4
Effect of ginsenoside Rd on IL-6 content in lung tissues of influenza
virus-infected mice (x̅ ± s, n = 10)

| | Lung tissue IL-6 (pg/mL) | | | |
|---|---|---|---|---|
| Group | 1 d | 3 d | 5 d | 7 d |
| Blank group | 41.39 ± 2.38 | 41.02 ± 2.21 | 41.15 ± 3.07 | 41.42 ± 2.67 |
| Model group | 49.08 ± 2.56 | 49.94 ± 3.29 | 48.89 ± 2.53 | 48.01 ± 3.11 |
| Ginsenoside Rd | 46.86 ± 2.76 | 46.29 ± 2.05 | 46.21 ± 2.73 | 46.52 ± 3.02 |
| low-dose | | | | |
| group | | | | |
| Ginsenoside Rd | 43.56 ± 3.46 | 43.47 ± 2.58 | 42.39 ± 3.41 | 42.21 ± 2.64 |
| high-dose | | | | |
| group | | | | |

The results showed that inflammatory factors TNF-α and IL-6 increased to varying degrees after influenza virus infection in mice, and the ginsenoside Rd could down-regulate the related inflammatory factors, and had an anti-inflammatory mechanism that may be related to the regulation of inflammatory factor secretion.

### Experimental Example 3 Effect of Ginsenoside Rd on Pneumonia Caused by Delta Variant of Novel Coronavirus in Mice

### 1.1 Experimental Animals and Pharmaceuticals

### 1.1.1 Experimental Animals:

hACE2 mice, aged 6-7 weeks and weighing 20-40 g, laboratory animal supplier: Jiangsu Gempharmatech Co., Ltd., laboratory animal production license: SCXK (Jiangsu) 2018-0008, and feed supplier: Jiangsu Medicience Biopharmaceutical Co., Ltd.

### 1.1.2 Virus and Drug:

COVID-19 Delta virus (BSL-3 Laboratory, Guangzhou Customs Technology Center (Laboratory of Highly Pathogenic Microorganisms, State Key Laboratory of Respiratory Diseases).

Ginsenoside Rd (content ≥95%), produced by Dalian Fusheng Natural Medicine Development Co., Ltd., detected by HPLC with Ginsenoside Rd provided by China Institute for Food and Drug Control as a reference substance, and having a content of 96.2%.

Dexamethasone, purchased from Shanghai Chemical Reagent Company.

### 1.2 Experimental Method

1.2.1 hACE2 transgenic C57BL/6 mice were divided into a normal group, a COVID-19 Delta virus infection group, drug groups (a 10 mg/kg low-dose group, and a 20 mg/kg high-dose group), and a positive control group (Dexamethasone, 5 mg/kg), with eight mice in each group. Except for the mice in the normal group who were administrated with PBS by nasal drops, the mice in the other groups were infected with COVID-19 Delta virus in 10⁵ PFU by nasal drops. 2 h after infection, the mice in each drug group were administrated with the drug by gavage for five consecutive days, once a day. Dead or dying animals were dissected and lungs were picked after euthanasia; and surviving animals were dissected on the Day 5 after infection, and lungs were picked.

1.2.2 In the infection group, four animals in each group were dissected for lung tissues for pathological study. Four animals were dissected, and lung tissues were picked. Total RNA was extracted from a homogenate supernatant of the lung tissues by a Trizol method, and inflammatory factor interleukin 16 (IL-1β) and monocyte chemoattractant protein 1 (MCP-1) were detected by RT-qPCR.

1.2.3 The specific operation steps of histopathological study of lung tissues were as follows:
(1) fixation and embedding: fixation (fixed with 4% paraformaldehyde)→washing and trimming, trimming tissue to obtain a flat section→dehydration with 50%, 70%, 80%, 95% and 100% ethanol solutions→immersion in xylene I and xylene II respectively for transparentizing→immersion with 60° C. wax I and II respectively for wax penetration→paraffin embedding; and
(2) slice staining: sectioning (a thickness of about 4 um)→immersion of paraffin section in a clarifying agent I, a clarifying agent II, and a clarifying agent III for deparaffinization→soaking in 100%, 90%, 80%, 70% and other levels of alcohol solutions for 5 min each→washing with water→staining with hematoxylin for 3 min→washing with water→differentiating with 0.5% hydrochloric acid alcohol for a few seconds→washing with tap water for 15 min for bluing→staining with eosin for 2 min→immersion in 95% ethanol I, 95% ethanol II, 100% ethanol I, 100% ethanol II, a clarifying agent I, a clarifying agent II, and a clarifying agent III respectively for dehydration and transparentizing→sealing with neutral gum for storage→observing and photographing under a microscope.

### 1.3. Test Results

(1) The pathological results of the lungs of the mice showed that compared with the normal group, the lung tissues of the infected model group presented pathological changes such as pulmonary hemorrhage and interstitial pneumonia. The specific types and degrees of lesions of infected animals in various groups were as follows:

TABLE 3-1
Pathomorphological observation results of lungs
of mice infected with COVID-19 Delta virus

| | Pulmonary hemorrhage | | | Interstitial pneumonia | | |
|---|---|---|---|---|---|---|
| | | Mod- | | | Mod- | |
| Group | Mild | erate | Severe | Mild | erate | Severe |
| Virus group | 0 | 4 | 0 | 0 | 3 | 1 |
| Dexamethasone | 3 | 1 | 0 | 3 | 1 | 0 |
| group | | | | | | |
| Ginsenoside Rd | 2 | 2 | 0 | 2 | 2 | 0 |
| low-dose group | | | | | | |
| Ginsenoside Rd | 4 | 0 | 0 | 4 | 0 | 0 |
| high-dose group | | | | | | |

Based on the comparison of the above lesions in various groups, the pathological changes of the surviving animals in various groups were milder than those of the dead animals; lesion degrees of the drug low-dose group and the drug high-dose group were milder than those of a virus group, and 10 mg/kg and 20 mg/kg drugs had good improvement effects on lung lesions caused by COVID-19 Delta virus; and the drug administrated to the high-dose group was better than Dexamethasone in the pathological improvement of pulmonary hemorrhage and interstitial pneumonia.
(2) The mRNA expression results of lung inflammatory factors showed that on the Day 5 of COVID-19 Delta virus infection for mice, the expressions of IL-1β and MCP-1 in a virus model group were significantly higher than that in the normal group; compared with the virus model group, IL-1β and MCP-1 inflammatory factors in the ginsenoside Rd drug group were significantly reduced; and the ability to inhibit the overexpression of inflammatory mediators in a 20 mg/kg administration group was considerable to that of Dexamethasone.

TABLE 3-2
Expression levels of IL-1β and MCP-1
in lung homogenates of COVID-19 Delta virus-
infected mice (x̅ ± s)

| Group | Dose | IL-1β | MCP-1 |
|---|---|---|---|
| Blank group | — | 1.00 ± 0.000 | 1.00 ± 0.000 |
| Model group | — | 46.52 ± 9.34 | 14.98 ± 11.35 |
| Dexamethasone | 5 mg/kg | 9.26 ± 10.27 | 6.27 ± 8.20 |
| group | | | |
| Ginsenoside Rd | 20 mg/kg | 10.13 ± 6.68 | 7.15 ± 9.36 |
| high-dose | | | |
| group | | | |
| Ginsenoside Rd | 10 mg/kg | 20.88 ± 6.42 | 18.71 ± 13.75 |
| low-dose | | | |
| group | | | |

### Experimental Example 4 Effect of Ginsenoside Rd on Inflammatory Response in Mice with Acute Lung Injury

### 1.1 Experimental Animals and Pharmaceuticals

1.1.1 Experimental animals:

Balb/c mice, male, weighing 20-22 g, were purchased from Liaoning Changsheng Biotechnology Co., Ltd., with quality certificate number: SCXK-(Liaoning) 2020-0001.

### 1.1.2 Reagents and Tested Drugs:

propylene glycol, purchased from Shanghai Macklin Biochemical Technology Co., Ltd.;
normal saline, purchased from Kunming Nanjiang Pharmaceutical Co., Ltd.;
lipopolysaccharide, Dexamethasone purchased from Sigma Corp. of America;
pentobarbital sodium, purchased from Shanghai Chemical Reagent Company;
enzyme-linked immunosorbent assay kit, mouse TNF-α detection kit, mouse IL-1β detection kit, mouse IL-6 detection kit, and mouse IL-18 detection kit, purchased from NanJing JianCheng Bioengineering Institute; and
test drug: ginsenoside Rd (content ≥95%), produced by Dalian Fusheng Natural Medicine Development Co., Ltd., detected by HPLC with Ginsenoside Rd provided by China Institute for Food and Drug Control as a reference substance, and having a content of 96.2%.

### 1.2 Experimental Method

### 1.2.1 Experimental Grouping and Administration

Balb/c male mice, weighing 20-22 g, were randomly divided into five groups according to body weights, with eight mice in each group: a blank control group (propylene glycol-0.5% carboxymethylcellulose sodium), a model group (LPS, 15 mg/kg), a ginsenoside Rd low-dose group (10 mg/kg), a ginsenoside Rd high-dose group (20 mg/kg), and a Dexamethasone group (5 mg/kg). Mice in each group were intraperitoneally injected; after 1 h of administration, except for the blank control group, the mice in each group were intratracheally instilled with 20 μg of LPS for modeling; and the blank control group was intratracheally instilled with an equal volume of PBS.

### 1.2.2 Establishment of Mouse ALI Model

After 1 h of administration, a mouse ALI model was established; each mouse was intraperitoneally injected with sodium pentobarbital at 30 mg/kg, anesthetized and fixed on an operating table dorsally; skin preparation was carried out on the neck; the skin was incised to expose the trachea; 20 μg of LPS was instilled into the trachea using a microglass syringe; and then the mouse was gently rotated head up on the operating table, such that the instilled liquid was evenly distributed in the mouse lungs, and the skin incision was sutured.

1.2.3 Determination of TNF-α, IL-1β, IL-6 and IL-18 content in lung tissues and BLAF

After 24 h of modeling, anesthetized mice were intraperitoneally injected with sodium pentobarbital at 30 mg/kg; lung tissues of the mice were taken and rinsed repeatedly in pre-cooled normal saline; the lung tissues were blood-removed, wiped with filter paper, weighed, and added with normal saline according to a ratio of 1:9 (W:V=1 g:9 ml); a tissue homogenate was prepared with a homogenization machine under an ice water bath; and a supernatant of mouse lung tissue homogenate was taken.

The mouse was anesthetized, and the trachea was separated; the whole lung was lavaged with 0.5 ml of pre-cooled normal saline, stayed for 1 min, and slowly pumped back with a syringe, and this process was repeated three times; and BLAF was collected, and centrifuged for 15 min at 4° C. and 1600 rpm/min.

In strict accordance with the operating instructions of an enzyme-linked immunosorbent assay kit (ELISA), the contents of TNF-α, IL-1β, IL-6 and IL-18 in the supernatant of mouse lung tissue homogenate and the BLAF supernatant were detected.

### 1.3 Results

Compared with the blank control group, the contents of TNF-α, IL-1β, IL-6 and IL-18 in lung tissues and alveolar lavage fluid of each mouse in the model group were significantly increased (P<0.01); and compared with the model group, the ginsenoside Rd drug group and the Dexamethasone group could significantly reduce the contents of TNF-α, IL-1β, IL-6 and IL-18 in the lung tissues and BLAF of the mice, and the effect of the ginsenoside Rd high-dose group was better than that of Dexamethasone (P<0.01).

TABLE 4-1
Effects of ginsenoside Rd on the contents of inflammatory factors in lung
tissue of mice with acute lung injury (X ± SD, n = 8)

| | | TNF-α | IL-1β | IL-6 | IL-18 |
|---|---|---|---|---|---|
| Group | Dose | (ng/L) | (ng/L) | (ng/L) | (ng/L) |
| Blank group | — | 39.67 ± 12.05 | 31.03 ± 8.62 | 28.62 ± 2.03 | 28.00 ± 6.35 |
| Model group | — | 115.26 ± 20.25 | 103.33 ± 23.26 | 101.96 ± 14.89 | 117.28 ± 21.22 |
| Ginsenoside Rd | 10 mg/kg | 98.14 ± 14.48 | 80.23 ± 18.08 | 84.70 ± 10.84 | 94.03 ± 15.22 |
| low-dose | | | | | |
| group | | | | | |
| Ginsenoside Rd | 20 mg/kg | 46.88 ± 19.52** | 30.52 ± 13.42** | 36.81 ± 13.45** | 37.54 ± 15.80* |
| high-dose | | | | | |
| group | | | | | |
| Dexamethasone | 5 mg/kg | 58.64 ± 10.65 | 48.76 ± 12.76 | 49.73 ± 16.08 | 49.06 ± 15.41 |
| group | | | | | |

Compared with a positive drug group,
**P < 0.01,
*P < 0.05

TABLE 4-2
Effects of ginsenoside Rd on the contents of inflammatory factors in alveolar
lavage fluid of mice with acute lung injury (X ± SD, n = 8)

| | | TNF-α | IL-1β | IL-6 | IL-18 |
|---|---|---|---|---|---|
| Group | Dose | (ng/L) | (ng/L) | (ng/L) | (ng/L) |
| Blank group | — | 159.64 ± 46.99 | 42.74 ± 13.67 | 21.34 ± 8.17 | 35.43 ± 8.35 |
| Model group | — | 475.22 ± 82.77 | 105.03 ± 25.52 | 55.03 ± 13.82 | 108.09 ± 34.55 |
| Ginsenoside Rd | 10 mg/kg | 344.09 ± 61.08 | 79.63 ± 21.33 | 39.37 ± 9.12 | 81.20 ± 21.27 |
| low-dose | | | | | |
| group | | | | | |
| Ginsenoside Rd | 20 mg/kg | 186.04 ± 75.00** | 49.51 ± 6.89** | 29.53 ± 8.99* | 44.64 ± 17.91** |
| high-dose | | | | | |
| group | | | | | |
| Dexamethasone | 5 mg/kg | 222.58 ± 60.91 | 58.56 ± 16.24 | 36.89 ± 6.16 | 56.38 ± 9.40 |
| group | | | | | |

Compared with a positive drug group,
**P < 0.01,
*P < 0.05

### Experimental Example 5 Effect of Ginsenoside Rd on Chronic Obstructive Pulmonary Disease in Rats

### 1.1 Experimental Animals and Pharmaceuticals

### 1.1.1 Experimental Animals:

SD rats, male, weighing 200±20 g, laboratory animal supplier: Hunan SJA Laboratory Animal Co., Ltd., laboratory animal production license: SCXK (Xiang) 2019-0004, and feed supplier: Jiangsu Medicience Biopharmaceutical Co., Ltd.

### 1.1.2 Drug:

Ginsenoside Rd (content ≥95%), produced by Dalian Fusheng Natural Medicine Development Co., Ltd., detected by HPLC with Ginsenoside Rd provided by China Institute for Food and Drug Control as a reference substance, and having a content of 96.2%.

Simvastatin tablets, produced by Zhejiang Jingxin Pharmaceutical Co., Ltd.

Dexamethasone, purchased from Shanghai Chemical Reagent Company.

Lipopolysaccharide (LPS, lot number: 08168), purchased from Sigma Corp.

TNF-α enzyme-linked immunosorbent assay kit, Nanjing Jiancheng Biotechnology Co., Ltd.

IL-8 ELISA assay kit, Nanjing Jiancheng Biotechnology Co., Ltd.

### 1.2 Experimental Method

### (1) Animal Grouping

After adaptive feeding (20-25° C., a humidity of 50-60%) for 1 week, the rats were randomly divided into a blank group, a model group, a positive control group, a ginsenoside Rd high-dose group, and a ginsenoside Rd low-dose group, with ten rats in each group.

### (2) Model Preparation

Blank group: standard feeding, no intervention.

Model group: on the Day 1 and Day 14, each rat was intraperitoneally injected with 10% chloral hydrate (300 mg/kg) for anesthesia; the head was fixed in a low position; the glottis was exposed; lipopolysaccharide was quickly injected into the trachea; and the rat was kept upright, and swung left and right for 10-20 s, such that the lipopolysaccharide was evenly distributed in the two lungs. On the Days 2-13, and 15-28, the lungs were smoked in a closed organic glass box for 30 min every day, with 15 cigarettes per day.

Positive control group: the animal was anesthetized, and intratracheally injected with lipopolysaccharide, wherein smoking conditions and time were the same as those of the model group; and after tracheal injection of lipopolysaccharide for two weeks, the animal was administrated with Simvastatin by gavage (2.5 mg/kg), and killed after six weeks, and serum was collected.

### (3) TNF-α and IL-8 Detection

In accordance with the instructions, the levels of TNF-α and IL-8 in the serum were detected by enzyme-linked immunosorbent assay. The data was expressed as x̅±s, p<0.05, which meant that it was statistically significant.

### 1.3. Test Results

### TNF-α and IL-8 Detection

The detection results of TNF-α and IL-8 showed that different concentrations of drugs intervened in both TNF-α and IL-6. The efficacy of the high-dose group was considerable to that of Simvastatin.

TABLE 5-1
Expression levels of TNF-α and IL-8 in serum of rats
(x̅ ± s, n = 10)

| | Group | TNF-α | IL-8 |
|---|---|---|---|
| Blank group | — | 9.04 ± 15.84 | 18.93 ± 12.73 |
| Model group | — | 13.78 ± 15.79 | 33.41 ± 8.92 |
| Simvastatin | 2.5 mg/kg | 11.71 ± 13.27 | 22.65 ± 11.56 |
| group | | | |
| Ginsenoside Rd | 10 mg/kg | 12.13 ± 17.06** | 24.88 ± 14.67** |
| high-dose | | | |
| group | | | |
| Ginsenoside Rd | 5 mg/kg | 12.92 ± 10.20 | 30.97 ± 13.70 |
| low-dose | | | |
| group | | | |

Compared with a positive drug group,
**P < 0.01

(2) The pathological results of the lungs of the rats showed that compared with the blank group, the lung tissues of the animals in the model group had obvious pathological changes.

TABLE 5-2
Pathomorphological observation results
of lungs of rats in experimental group

| | | | | Interstitial inflammatory | | |
|---|---|---|---|---|---|---|
| | Epithelial cell necrosis | | | cell infiltration | | |
| | | Mod- | | | Mod- | |
| Experimental group | Mild | erate | Severe | Mild | erate | Severe |
| Model group | 0 | 7 | 3 | 0 | 6 | 4 |
| Simvastatin group | 7 | 3 | 0 | 6 | 4 | 0 |
| Drug low-dose group | 6 | 4 | 0 | 6 | 4 | 0 |
| Drug high-dose group | 7 | 3 | 0 | 7 | 3 | 0 |

Based on the comparison of the above lesions in various groups, the lesion degrees of the drug low-dose group and the drug high-dose group were milder than that of the model group; 10 mg/kg and 5 mg/kg drugs had good improvement effects on lung lesions; and the high-dose group had an effect considerable to the Simvastatin group.

### Experimental Example 6 Inhibitory Effect of Ginsenoside Rd on Airway Inflammations in Mice with Chronic Bronchial Asthma

### 1.1 Experimental Animals and Pharmaceuticals

### 1.1.1 Experimental Animals:

BALB/c mice, female, weighing 22±2 g, laboratory animal supplier: Hunan SJA Laboratory Animal Co., Ltd., laboratory animal production license: SCXK (Xiang) 2019-0004, and feed supplier: Jiangsu Medicience Biopharmaceutical Co., Ltd.

### 1.1.2 Drug:

ginsenoside Rd (content ≥95%), produced by Dalian Fusheng Natural Medicine Development Co., Ltd., detected by HPLC with Ginsenoside Rd provided by China Institute for Food and Drug Control as a reference substance, and having a content of 96.2%.

Ovalbumin (OVA), purchased from Sigma, Corp.

TNF-γ enzyme-linked immunosorbent assay kit, Nanjing Jiancheng Biotechnology Co., Ltd.

IL-4 ELISA assay kit, Nanjing Jiancheng Biotechnology Co., Ltd.

### 1.2 Experimental Method

### (1) Animal Grouping

After adaptive feeding (20-25° C., a humidity of 50-60%) for one week, the mice were randomly divided into a blank group, a model group, a ginsenoside Rd high-dose group, and a ginsenoside Rd low-dose group, with ten rats in each group.

### (2) Model Preparation

Except for the blank group, the mice in the other groups were intraperitoneally injected with 0.2 ml of sensitization solution (containing 50 μg of OVA) on the Day 1, Day 8 and Day 15, respectively. Starting on Day 22, the mice in each group were placed in a nebulizer and inhaled with 2% OVA for exciting asthma. This inhalation was performed once a day, 45 min each time, for seven consecutive days.

Normal saline was used in the blank group instead of OVA.

### (3) Administration Method

Administration by gavage was performed 30 min before each asthma stimulation, once a day for seven consecutive days. The blank group and the model group were administrated with normal saline by gavage, the high-dose group was administrated with 10 mg/kg ginsenoside Rd by gavage, and the low-dose group was administrated with 5 mg/kg ginsenoside Rd by gavage.

### (4) Detection:

24 h after the last excitation, the mice were sampled, blood was collected from the eyeballs and separated to obtain serum, and IL-4 and INF-γ in the serum were detected.

### 1.3. Test Results

### (1) IL-4 and INF-γ Detection Results

TABLE 6
Concentrations of IL-4 and INF-γ in serum of mice (x̅ ± s, n = 10)

| | n | IL-4 (pg/mL) | INF-γ |
|---|---|---|---|
| Blank group | 10 | 27.30 ± 2.84 | 104.30 ± 12.83 |
| Model group | 8 | 39.07 ± 4.73 | 90.45 ± 6.32 |
| Ginsenoside Rd | 10 | 36.11 ± 10.98** | 102.48 ± 7.81** |
| high-dose | | | |
| group | | | |
| Ginsenoside Rd | 8 | 10.72 ± 16.74 | 95.34 ± 13.59 |
| low-dose | | | |
| group | | | |

The results showed that the ginsenoside Rd could increase the expression of INF-γ and reduce the expression of IL-4, thereby reducing airway inflammations in mice with chronic bronchial asthma and playing a role in the prevention and treatment of asthma.

We claim:

1. An application of ginsenoside Rd in preparation of a drug for relieving or/and treating acute or chronic inflammations or health food/medicinal and edible food/functional food/dietary supplement.

2. The application according to claim 1, wherein the acute inflammations include influenza virus pneumonia, novel coronavirus pneumonia, acute lung injury inflammations, and the like.

3. The application according to claim 2, wherein a coronavirus is a COVID-19 Delta virus.

4. The application according to claim 1, wherein the chronic inflammations include chronic obstructive pneumonia and chronic asthma airway inflammations.

5. The application according to claim 1, wherein the ginsenoside Rd is cited separately.

6. The application according to claim 1, wherein the relief or treatment effect is better than that of Dexamethasone.

7. The application according to claim 1, wherein the content of the ginsenoside Rd in the drug or health food/functional food/dietary supplement is 1% to 99.9%.

8. The application according to claim 1, wherein the ginsenoside Rd can be extracted and separated from roots, stems, leaves, flowers and fruits of Radix Notoginseng, Radix Ginseng, Radix Panacis Quinquefolii or other plants, and can also be prepared by using processes such as chemical synthesis, chemical transformation and biological transformation.

9. The application according to claim 1, wherein the drug or health food/functional food/dietary supplement is composed of the ginsenoside Rd and a pharmaceutically acceptable carrier or a food-acceptable carrier.

10. The application according to claim 1, wherein the drug or health food/medicinal and edible food/functional food/dietary supplement further contains one or more of a disintegrant, a humectant, a binder, filler, an absorption promoter, a solvent, a lubricant, a surfactant, a flavoring agent, a sweetener, an antioxidant, a preservative and pigment, an ointment base, a transdermal promoter, etc.

11. The application according to claim 1, wherein the drug or health food/medicinal and edible food/functional food/dietary supplement further comprises one or more of a Radix Scrophulariae extract, a Rehmannia glutinosa Libosch extract, a Radix Ophiopogonis extract, a Radix Scutellariae extract, a Radix Ranunculi Ternati extract, a Fritillaria extract, a Menthae Haplocalycis Herba extract, a Semen Sesami Nigrum extract, a Zingiber officinale Roscoe extract, a grape seed extract, a pomegranate seed extract, a Rhizoma Cyperi extract, a Pericarpium Citri Reticulatae Viride extract, plant essential oil, arbutin, vitamin C and derivatives thereof, or vitamin E and derivatives thereof.

12. The application according to claim 9, wherein a ratio of a weight of the ginsenoside Rd to a total weight of the drug or health food/medicinal and edible food/functional food/dietary supplement is (0.01-100): 100.

13. The application according to claim 1, wherein the drug is an oral preparation, a topical preparation or an injection; preferably, the oral preparation is tablets, capsules, granules, pills, a solution, a suspension, an emulsion, an oral liquid, drops, or a syrup; preferably, the topical preparation is emplastrum, an ointment or a suppository; and preferably, the injection is an injection solution, a powder injection or a lyophilized powder injection.

14. The application according to claim 1, wherein the health food/medicine and edible food/functional food/dietary supplement is an oral preparation; and preferably, the oral preparation is tablets, a powder, granules, medicinal tea, pills, a paste, hard capsules, soft capsules, an oral liquid, medicated wine, a syrup, or drops.
